(19) United States
(12) Patent Application Publication
(10) Pub. No.: US 2010/0042143 A1
(43) Pub. Date: Feb. 18, 2010

(54) Method of Transferring Pressure in an Articulating Surgical Instrument

(76) Inventor: James S. Cunningham, Boulder, CO (US)

Correspondence Address: TYCO Healthcare Group LP;Attn: IP Legal, 5920 Longbow Drive, Mail Stop A36, Boulder, CO, 80301-3299, US

(21) Appl. No.: 12/192,189

(22) Filed: Aug. 15, 2008

(51) Int. Cl. A61B 17/29

(52) U.S. Cl. 606/208

(57) ABSTRACT

An end effector for a surgical instrument includes a fixed bearing member with mounting surfaces for attachment to a distal end of the surgical instrument. At least one jaw member of the end effector is configured to move relative to an opposing jaw member between open and closed configurations, and a force transfer member is configured for longitudinal motion with respect to the fixed bearing member. The end effector also includes a reactive member that has a pivot boss about which the at least one jaw member rotates, and is coupled between the fixed bearing member and the at least one jaw member. The force transfer member applies a longitudinal force to the at least one jaw member at some lateral distance from the pivot boss to urge the at least one jaw member to move between the open and closed configurations.

### BACKGROUND

1. Technical Field

The present disclosure relates to an apparatus for remotely activating jaw members on an articulating surgical instrument. In particular, the apparatus provides an end effector capable of transferring a sufficient force to the jaw members to cause a therapeutic effect on tissue clamped between the jaw members.

2. Background of Related Art

Typically in a laparoscopic, an endoscopic, or other minimally invasive surgical procedure, a small incision or puncture is made in a patient's body. A cannula is then inserted into a body cavity through the incision, which provides a passageway for inserting various surgical devices such as scissors, dissectors, retractors, or similar instruments. To facilitate operability through the cannula, instruments adapted for laparoscopic surgery typically include a relatively narrow shaft supporting an end effector at its distal end and a handle at its proximal end. Arranging the shaft of such an instrument through the cannula allows a surgeon to manipulate the proximal handle from outside the body to cause the distal end effector to carry out a surgical procedure at a remote internal surgical site. This type of laparoscopic procedure has proven beneficial over traditional open surgery due to reduced trauma, improved healing and other attendant advantages.

An articulating laparoscopic or endoscopic instrument may provide a surgeon with a range of operability suitable for a particular surgical procedure. The instrument may be configured such that the end effector may be aligned with an axis of the instrument to facilitate insertion through a cannula, and thereafter, the end effector may be caused to articulate, pivot or move off-axis as necessary to appropriately engage tissue. When the end effector of an articulating instrument comprises a pair of jaw members for grasping tissue, a force transmission mechanism such as a flexible control wire may be provided to open or close the jaws. For example, the control wire may extend through an outer shaft from the handle to the jaws such that the surgeon may create a tension in the control wire to cause the jaws to move closer to one another. The closure or clamping force generated in the jaws may be directly related to the tension in the control wire applied by the surgeon.

One type of laparoscopic or endoscopic instrument is intended to generate a significant closure force between jaw members to seal small diameter blood vessels, vascular bundles or any two layers of tissue with the application electrosurgical or RF energy. The two layers may be grasped and clamped together by the jaws of an electrosurgical forceps, and an appropriate amount of electrosurgical energy may be applied through the jaws. In this way, the two layers of tissue may be fused together. The closure forces typically generated by this type of procedure may present difficulties when using a typical control wire to open and close the jaws of an articulating instrument.

For example, a surgeon's efforts to position the jaws may be frustrated by a tendency for a control wire under tension to realign the jaws with the axis of the instrument after the jaws have been articulated off-axis. Although this tendency may be observed in any type of articulating instrument, the tendency is particularly apparent when the closure forces and necessary tension in the control wire are relatively high, as is common in an electrosurgical sealing instrument. This tendency may be created by the direction of reaction forces through the outer shaft of the instrument.

### SUMMARY

The present disclosure describes an end effector for incorporation into an articulating surgical instrument, which decouples a force application mechanism from an outer shaft of the instrument. The end effector includes a fixed bearing member, which defines an end effector axis and provides mounting surfaces for attachment to a distal end of the surgical instrument. The end effector also includes at least one jaw member that is configured to move relative to an opposing jaw member between an open configuration and a closed configuration. A force transfer member is configured for longitudinal motion with respect to the fixed bearing member, and a reactive member is coupled between the fixed bearing member and the at least one jaw member. The reactive member includes a pivot boss about which the at least one jaw member rotates as it moves between the open configuration and the closed configuration. The force transfer member is configured to contact the at least one jaw member such that longitudinal motion of the force transfer member applies a force to the at least one jaw member at some lateral distance from the pivot boss to urge the at least one jaw member to move relative to the opposing jaw member between the open configuration and the closed configuration.

The end effector may further include a motion conversion mechanism operatively associated with the force transfer member to urge the force transfer member longitudinally. The motion conversion mechanism may include an input shaft configured for rotational movement relative to the fixed bearing member, and the input shaft may be further configured for connection to a torsion cable or rod to receive rotational motion therefrom. The input shaft may be coupled to a power screw and the force transfer member may be coupled to a translation nut such that the translation nut translates longitudinally upon rotational motion in the power screw. The motion conversion mechanism may also include a worm gear.

The force transfer member may be coupled to the at least one jaw member such that distal translation of the force transfer member moves the at least one jaw member to the closed configuration, and proximal translation of the force transfer member moves the at least one jaw member to the open configuration. The at least one jaw member may include a pair of moveable jaws, or the end effector may include an opposing jaw member that is stationary relative to the fixed bearing member.

According to another aspect of the disclosure a surgical instrument includes a handle portion near a proximal end of the surgical instrument adapted for manipulation by a user to control the surgical instrument, a tubular shaft extending distally from the handle portion and defining an instrument axis, and an end effector pivotally coupled to a distal end of the tubular shaft such that the end effector may articulate relative to the instrument axis. The end effector defines an end effector axis and includes a pair of jaw members configured to pivot about a pivot axis that is transverse to the end effector axis to move between an open and a closed configuration. The end effector also includes a force transfer member configured for longitudinal motion with respect to a fixed member in a direction along the end effector axis. The force transfer member is configured to contact at least one of the jaw members of the pair of jaw members at some lateral distance from the pivot axis and transfer a longitudinal force thereto when the pair of jaws is in the closed configuration. The end effector also includes a reactive member coupled to the fixed member and to the at least one jaw member of the pair of jaw members such that a reactionary force resulting from the force transferred to the at least one jaw member of the pair of jaw members is realized in the reactive member. The reactive member includes a pivot boss about which the at least one of the jaw members pivots.

The end effector may further include a motion conversion mechanism operatively associated with the force transfer member to urge the force transfer member longitudinally. Also, a torsion cable or rod may be coupled to the end effector to deliver rotational motion thereto.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1A is a perspective view of an articulating laparoscopic surgical instrument that may incorporate the features of the present disclosure;

FIG. 1B is a perspective view of an embodiment of an articulating surgical instrument according to one embodiment of the present disclosure;

FIG. 2A is a perspective view of an end effector in accordance with an embodiment of the present disclosure in an open configuration;

FIG. 2B is a perspective view of the end effector of FIG. 2A in a closed configuration;

FIG. 3 is a top view of the end effector of FIG. 2A in the open configuration;

FIG. 4A is a side view of the end effector of FIG. 2A in the open configuration;

FIG. 4B is a side view of the end effector of FIG. 2A in the closed configuration;

FIG. 5A is an enlarged, side view of a pivoting portion of the end effector of FIG. 2A in a nearly closed configuration;

FIG. 5B is an enlarged, side view of the pivoting portion of the end effector of FIG. 2A in the closed configuration;

FIG. 6A is a partial top view of an alternate embodiment of an end effector in accordance with the present disclosure;

FIG. 6B is a side view of the end effector of FIG. 6A;

FIG. 7A is a top view of another alternate embodiment of an end effector in accordance with the present disclosure;

FIG. 7B is a side view of the end effector of FIG. 7A in an open configuration; and

FIG. 7C is a side view of the end effector of FIG. 7A in a closed configuration.

### DETAILED DESCRIPTION

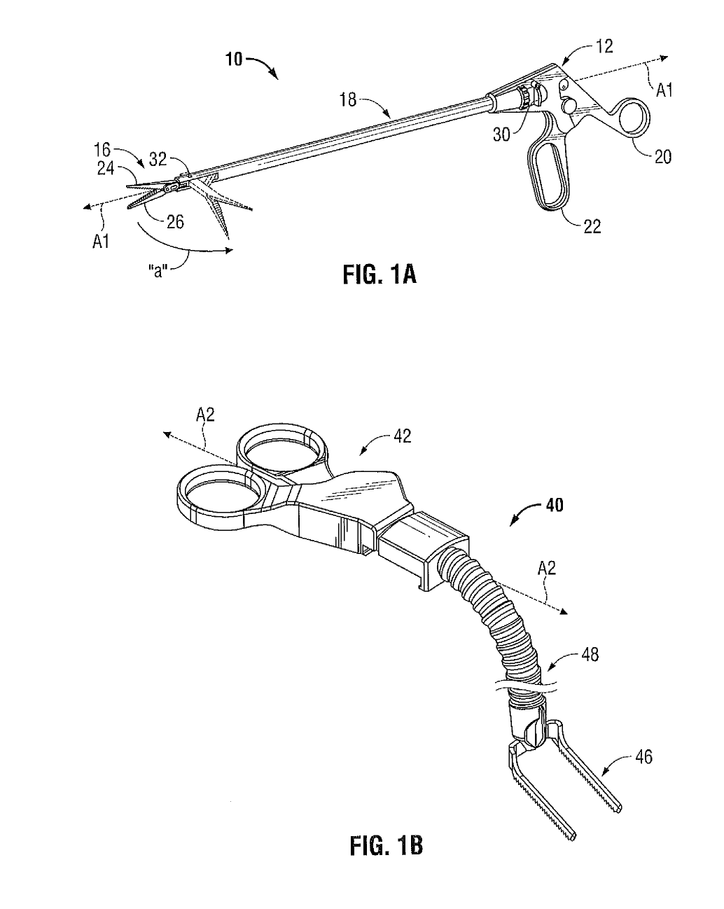

Referring initially to FIG. 1A, an articulating endoscopic instrument is depicted generally as 10. The instrument 10 includes a handle portion 12 near a proximal end, an end effector 16 near a distal end and an elongated shaft 18 therebetween. Elongated shaft 18 defines an instrument axis “A1” to which end effector 16 aligns for insertion through a cannula (not shown) or other suitable introducer. End effector 16 is articulatable off-axis (as indicated in phantom) to appropriately engage tissue. Handle portion 12 is manipulatable by the surgeon from outside a body cavity to control the movement of the end effector 16 positioned inside the body at a tissue site. For example, the surgeon may separate and approximate a pivoting handle 20 relative to a stationary handle 22 to respectively open and close jaw members 24, 26. Also, a surgeon may pivot lever 30 to cause the end effector 16 to articulate or pivot in a horizontal plane about a pivot pin 32. A more complete description of the components and operation of instrument 10 may be found in U.S. Patent Application Publication No. 2006/0025907 to Nicholas et al.

Another type of known articulating surgical instrument is depicted generally as 40 in FIG. 1B. Instrument 40 includes a handle portion 42 that is manipulatabe to control the movement of end effector 46. Handle portion 42 is coupled to end effector 46 through a flexible shaft 48 that moves into and out of alignment with instrument axis “A2.”

Both articulating instruments 10, 40 provide for off-axis operation of the respective end effectors 16, 46. Both instruments 10, 40 may exhibit a tendency to align themselves to the respective instrument axes A1, A2 when the end effectors 16, 46 are operated if the instruments 10, 40 are equipped with a force transmission mechanism that generates reaction forces in outer shafts 18, 48. Accordingly, an end effector 100 as described below may be incorporated into instruments similar to instruments 10, 40 to decouple any reactionary forces from outer shafts of the instruments. End effectors in accordance with the present disclosure may also be incorporated into a non-articulating instrument.

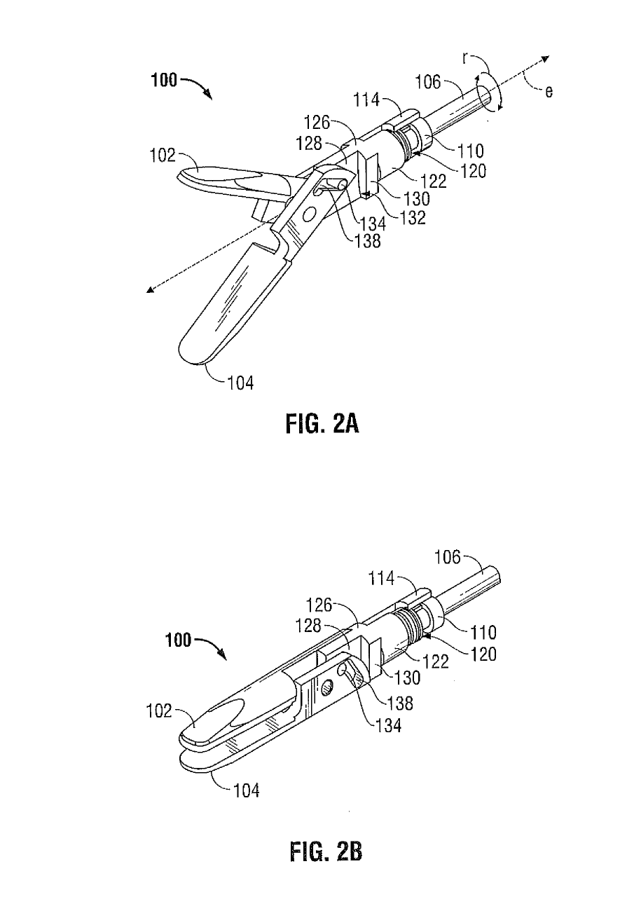

Referring now to FIGS. 2A through 5B, an end effector in accordance with the present disclosure is depicted generally as 100. End effector 100 includes jaw members 102 and 104 that are selectively movable between an open configuration as seen in FIG. 2A and a closed configuration as depicted in FIG. 2B. This motion of the jaw members 102, 104 is achieved upon the application of a torsion force to end effector 100. Therefore, a control wire placed in tension, which as discussed above may generate reactionary forces in the outer shaft of an instrument and tend to frustrate the articulation of the instrument, is not necessary.

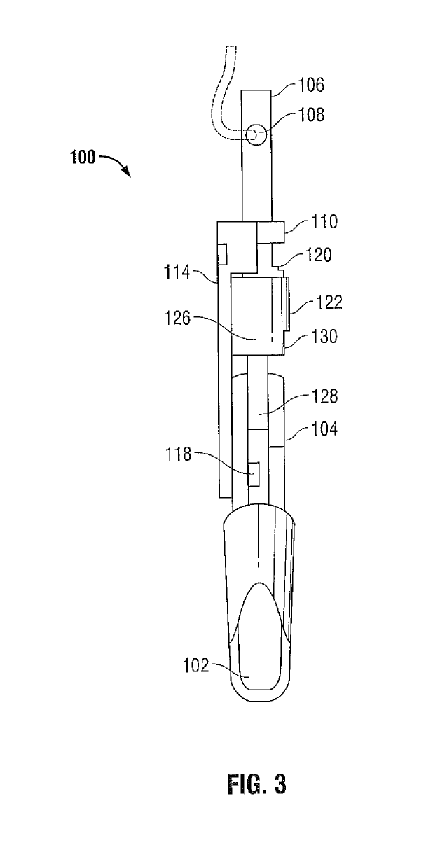

End effector 100 is adapted to receive a torsion force through input shaft 106 such that input shaft 106 may rotate about an end effector axis “e” as indicated by arrows “r.” Input shaft 106 includes a bore 108 (FIG. 3), which provides connectivity to a suitable external source of rotational motion (not shown). The rotational motion may be generated, for example, by an electric motor, or alternatively by a surgeon using a manual control surface at a handle portion of the instrument. If the rotational motion is generated in a handle portion of the instrument, a flexible torsion cable (shown in phantom in FIG. 3) may be positioned through the instrument shaft to transmit rotational motion from the handle to the end effector 100.

Input shaft 106 rotates inside a fixed bearing member 110. Fixed bearing member 110 provides mounting surfaces for direct or indirect fixed coupling to an articulating distal end of an instrument shaft, which remains stationary relative thereto. In this way, the entire end effector 100 is supported by the instrument and may be caused to articulate relative to an instrument axis. Fixed bearing member 110 also supports a reactive member 114 on an outer surface thereof. As best seen in FIG. 3, reactive member 114 extends distally from fixed bearing member 110 and comprises a pivot boss 118 (FIG. 3) extending into jaw member 102. Jaw member 102 is pivotable about pivot boss 118 as the end effector 100 is moved between the open and closed configurations. Although removed from the figures for clarity, an additional reactive member 114 is supported by fixed bearing member 110 so as to mirror the reactive member 114 shown and provide a pivot boss 118 about which jaw member 104 may rotate when end effector 100 is moved between the open and closed configurations. Reactive member 114 remains stationary relative to fixed bearing member 110 as jaw members 102, 104 pivot open and closed.

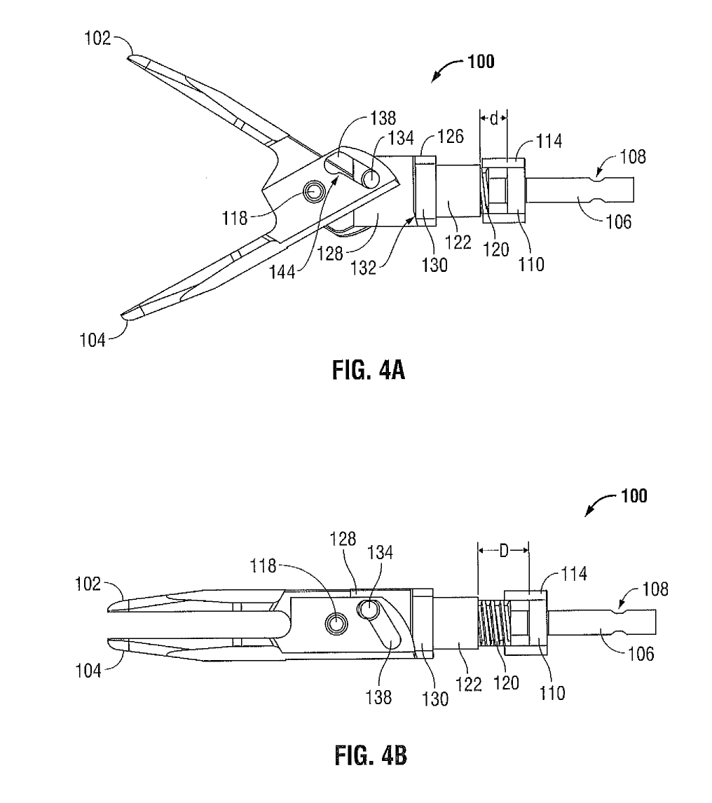

A power screw 120 is supported at a distal end of input shaft 106. The power screw 120 is coupled to the input shaft 106 such that both the power screw 120 and the input shaft 106 rotate together. Rotation of the power screw 120 drives a translation nut 122 longitudinally along end effector axis “e.” For example, rotation of power screw 120 in a first direction advances translation nut 122 from the position depicted in FIG. 4A where the translation nut is disposed at a distance “d” from the fixed bearing member 110, to the position depicted in FIG. 4B where the translation nut 122 is a greater distance “D” from the fixed bearing member 110. Likewise, rotation of power screw 120 in an opposite direction withdraws translation nut 122 such that translation nut 122 becomes closer to the fixed bearing member 110.

A force transfer member 126 is supported at a distal end of translation nut 122. Force transfer member 126 may be coupled to translation nut 122 or may be formed integrally therewith such that the force transfer member 126 translates along with the translation nut 122. Force transfer member 126 is formed with a central web 128 having a pair of proximal flanges 130 extending therefrom in opposite directions. The proximal flanges 130 exhibit sloped base portions 132 at their lower ends. An opposed pair of cam pins 134 also protrudes from central web 128.

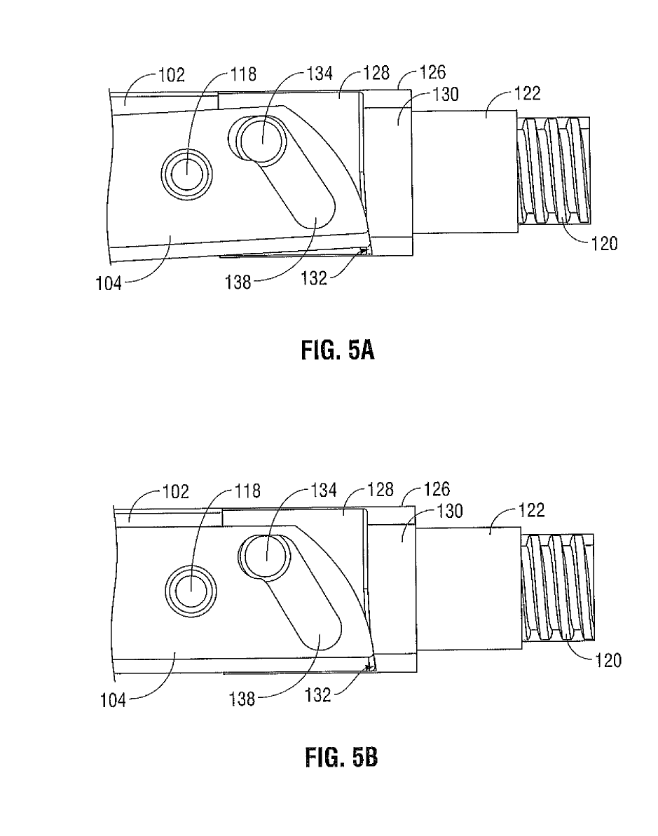

The cam pins 134 work in conjunction with proximal flanges 130 to open and close the jaw members 102, 104. Cam pins 134 engage a pair of cam slots 138 on the jaw members 102, 104 as the cam pins 134 translate distally along with force transfer member 126. Distal translation of cam pins 134 through cam slots 138 cause the jaw members 102, 104 to move from the open configuration of FIG. 4A to the nearly-closed configuration of FIG. 5A. In the nearly-closed configuration, the sloped base portions 132 of the proximal flanges 130 contact proximal faces of jaw members 102, 104. Also at the nearly closed configuration, each of the cam pins 134 reach a curve 144 in the respective cam slots 138 that allows force to be transferred from the cam pins 134 to the proximal flanges 130 of the force transfer member 126. Further distal translation of the force transfer member 126 will move the jaws from the nearly-closed configuration of FIG. 5A to the closed configuration of FIG. 5B as the sloped base portions 132 press against the proximal faces of the jaw members 102, 104.

In the closed configuration of FIGS. 2B, 4B and 5B, the jaw members 102, 104 may generate a significant clamping force that can be directed at tissue positioned between the jaw members 102, 104. As the proximal flanges 130 press distally against the jaw members 102, 104, the jaw members 102, 104 press distally on the pivot bosses 118 of reactive member 114. An opposite reaction force is realized as a tensile force in the reactive member 114, which links the jaw members to the fixed bearing member 110. Because the reaction force is contained entirely within the end effector 100, this arrangement allows an articulating instrument to which the end effector 100 is attached to close jaw members 102, 104 without creating a tendency for the end effector to conform to an axis of the instrument.

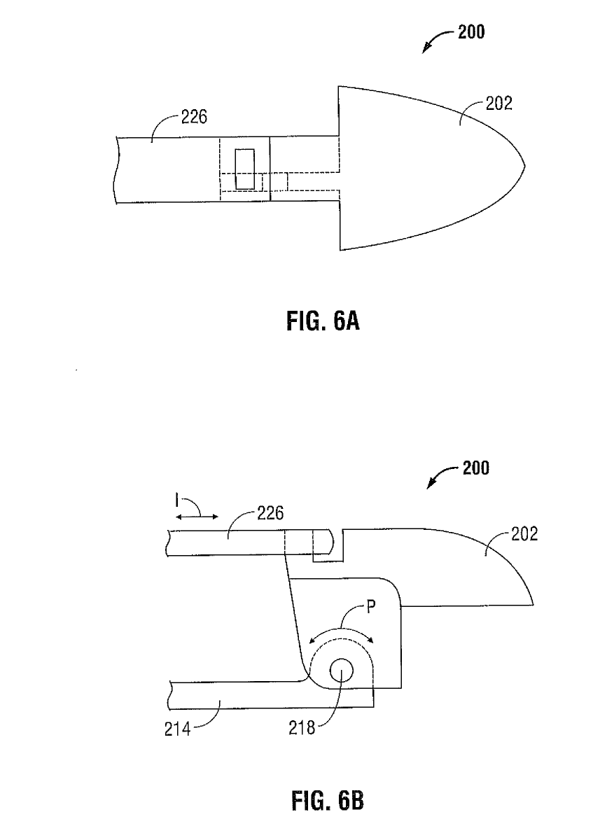

Referring now to FIGS. 6A and 6B, an alternate embodiment of an end effector in accordance with the present disclosure is depicted generally as 200. End effector 200 defines a lever cam arrangement and comprises a jaw member 202, a reactive member 214, which supports a pivot boss 218, and a force transfer member 226. Jaw member 202 is configured to pivot about pivot boss 218 (as indicated by arrows “p”) in response to longitudinal translation (as indicated by arrows “l”) of the force transfer member 226 at some lateral distance from the pivot boss 218. End effector 200 may be equipped with an opposing jaw member (not shown), stationary or moveable, such that jaw member 202 is moved between an open and closed configuration as it pivots about pivot boss 218. The force transfer member 226 is coupled to the jaw member 202 such that distal translation of the force transfer member 226 moves jaw member 202 to the closed configuration, and proximal translation of the force transfer member 226 moves jaw member 202 to the open configuration.

Reactive member 214 is supported at a proximal end by a fixed member (not shown) as part of a motion conversion mechanism that converts rotational motion to longitudinal motion. For example, a motion conversion mechanism may include an arrangement of a power screw and translation nut as described above. Alternatively, a worm gear arrangement may be configured to drive force transfer member 226 longitudinally relative to reactive member 214. This arrangement would also allow reactive member 214 to carry reactive forces entirely within the end effector 200. Reactive member 214, however, would be placed in compression as jaw member 202 is moved to the closed configuration.

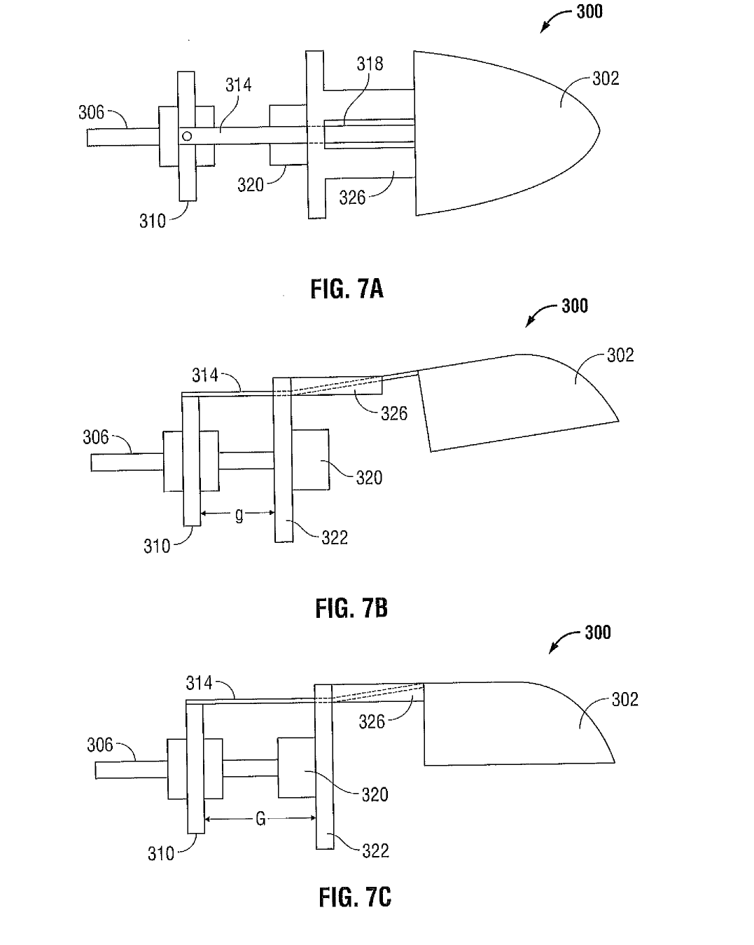

Referring now to FIGS. 7A through 7C, another alternate embodiment of an end effector in accordance with the present disclosure is depicted generally as 300. End effector 300 includes a jaw member 302, which is movable between an open configuration and a closed configuration as described below. End effector 300 is adapted to receive a torsion force from an external source through input shaft 306. Input shaft 306 rotates inside a fixed bearing member 310. Fixed bearing member 310 is coupled to an articulating distal end of an instrument shaft and remains stationary relative thereto. In this way, the entire end effector 300 is supported by the instrument and may be caused to articulate relative to an instrument axis.

Fixed bearing member 310 also supports a reactive member 314 on an upper surface thereof. Reactive member 314 is formed from a thin strip of conformable material such as spring steel or a shape memory alloy, and extends distally from fixed bearing member 310 to jaw member 302 through a pivot channel 318. Longitudinal motion of the reactive member 314 through the pivot channel 318 causes reactive member 314 to flex in an upward or downward direction to move jaw member 302 between an open configuration as depicted in FIG. 7B and a closed configuration as depicted in FIG. 7C.

A power screw 320 is supported at a distal end of input shaft 306 such that both the power screw 320 and the input shaft 306 may rotate together. Rotation of the power screw 320 drives a translation nut 322 longitudinally with respect to fixed bearing member 310. For example, rotation of power screw 320 in a first direction advances translation nut 322 from the position depicted in FIG. 7B where a gap “g” separates translation nut 322 from fixed bearing member 310, to the position depicted in FIG. 7C where a larger gap “G” separates translation nut 322 from fixed bearing member 310. Likewise, rotation of power screw 320 in an opposite direction withdraws translation nut 322 such that it becomes closer to the fixed bearing member 310.

A force transfer member 326 is supported at an upper end of translation nut 322. Force transfer member 326 may be coupled to translation nut 322 or formed integrally therewith such that the force transfer member 326 translates along with translation nut 322. Pivot channel 318 is extends entirely through force transfer member 326 at a distal end such that force transfer member 326 exhibits a forked configuration as best seen in FIG. 7A. When end effector 300 is in the closed configuration depicted in FIG. 7C, a distal end of the forked force transfer member 326 contacts a proximal face of the jaw member 302. This allows force to be transferred from the reactive member 314 to the force transfer member 326. Further distal translation of the translation nut 322 will result in force transfer member 326 pressing against the proximal face of the jaw member 302 such that jaw member 302 may generate a substantial clamping force. When the force transfer member 326 presses against the jaw member 302, a reaction force is realized as a tensile force in the reactive member 314. Since the reaction force is contained within the end effector 300, the closure of jaw member 302 does not tend to frustrate the articulation of an instrument to which end effector 300 is coupled.

Although the foregoing disclosure has been described in some detail by way of illustration and example, for purposes of clarity or understanding, it will be obvious that certain changes and modifications may be practiced within the scope of the appended claims.

What is claimed is:

1. An end effector for a surgical instrument, comprising:
a fixed bearing member defining a longitudinal axis and providing mounting surfaces for attachment to a distal end of the surgical instrument;
at least one jaw member configured to move relative to an opposing jaw member between an open configuration and a closed configuration;
a force transfer member configured for longitudinal motion with respect to the fixed bearing member; and
a reactive member including a pivot boss about which the at least one jaw member rotates as it moves between the open configuration and the closed configuration, the reactive member coupled between the fixed bearing member and the at least one jaw member;
wherein the force transfer member is configured to contact the at least one jaw member such that longitudinal motion of the force transfer member applies a force to the at least one jaw member at some lateral distance from the pivot boss to urge the at least one jaw member to move relative to the opposing jaw member between the open configuration and the closed configuration.

2. The end effector according to claim 1, further comprising a motion conversion mechanism operatively associated with the force transfer member to urge the force transfer member longitudinally.

3. The end effector according to claim 2, wherein the motion conversion mechanism includes an input shaft configured for rotational movement relative to the fixed bearing member, the input shaft further configured for connection to a torsion cable or rod to receive rotational motion therefrom.

4. The end effector according to claim 3, wherein the input shaft is coupled to a power screw and the force transfer member is coupled to a translation nut such that the translation nut translates longitudinally upon rotational motion in the power screw.

5. The end effector according to claim 2, wherein the wherein motion conversion mechanism comprises a worm gear.

6. The end effector according to claim 1, wherein the force transfer member is coupled to the at least one jaw member such that distal translation of the force transfer member moves the at least one jaw member to the closed configuration, and proximal translation of the force transfer member moves the at least one jaw member to the open configuration.

7. The end effector according to claim 1, wherein the at least one jaw member includes a pair of moveable jaws.

8. The end effector according to claim 1, wherein the opposing jaw member is stationary relative to the fixed bearing member.

9. A surgical instrument comprising:
a handle portion near a proximal end of the surgical instrument adapted for manipulation by a user to control the surgical instrument;
a tubular shaft extending distally from the handle portion and defining an instrument axis; and
an end effector pivotally coupled to a distal end of the tubular shaft such that the end effector may articulate relative to the instrument axis, the end effector defining an end effector axis and comprising:
a pair of jaw members configured to pivot about a pivot axis to move between an open and a closed configuration, the pivot axis transverse to the end effector axis;
a force transfer member configured for longitudinal motion with respect to a fixed member in a direction along the end effector axis, the force transfer member configured to contact at least one of the jaw members of the pair of jaw members at some lateral distance from the pivot axis and transfer a longitudinal force thereto when the pair of jaws is in the closed configuration; and
a reactive member coupled to the fixed member and to the at least one of the jaw members of the pair of jaw members such that a reactionary force resulting from the force transferred to the at least one jaw member of the pair of jaw members is realized in the reactive member, the reactive member including a pivot boss about which the at least one of the jaw members pivots.

10. The surgical instrument according to claim 9, wherein the end effector further comprises a motion conversion mechanism operatively associated with the force transfer member to urge the force transfer member longitudinally.

11. The surgical instrument according to claim 10, further comprising a torsion cable or rod coupled to end effector to deliver rotational motion thereto.
